(19) United States
(12) Patent Application Publication
(10) Pub. No.: US 2019/0211305 A1
(43) Pub. Date: Jul. 11, 2019

(54) Method for Culturing Pluripotent Stem Cells on Specific Laminin

(71) Applicants: Kyoto University, Kyoto (JP); Osaka University, Osaka (JP); Megakaryon Corporation, Kyoto (JP)

(72) Inventors: Koji Eto, Kyoto (JP); Sou Nakamura, Kyoto (JP); Kiyotoshi Sekiguchi, Osaka (JP); Tomohiro Shigemori, Kyoto (JP)

(21) Appl. No.: 16/326,841

(22) PCT Filed: Aug. 25, 2017
(86) PCT No.: PCT/JP2017/030467

§ 371 Date: Feb. 20, 2019

(30) Foreign Application Priority Data
Aug. 25, 2016 (JP) 2016-164597

(51) Int. Cl. C12N 5/0735; C12N 5/077; C12N 5/071; C12N 5/078

(52) U.S. Cl. CPC C12N 5/0606; C12N 5/0658; C12N 5/0687; C12N 5/0657; C12N 2501/15; C12N 2533/52; C12N 2501/115; C12N 2501/165; C12N 5/0634

(57) ABSTRACT

The present invention provides a method for culturing pluripotent stem cells, comprising the step of contacting pluripotent stem cells with laminin 421 or a fragment thereof, laminin 121 or a fragment thereof, or a combination thereof.

### TECHNICAL FIELD

The present invention relates to a novel method for culturing pluripotent stem cells using a specific laminin, and more particularly, a culturing method for preparing pluripotent stem cells that easily differentiate into mesodermal cells.

### BACKGROUND ART

Since pluripotent stem cells such as ES cells or iPS cells are able to proliferate indefinitely while retaining pluripotency, a required number of these cells for use in transplantation can be easily obtained. Consequently, these cells are attracting attention as raw materials of cell transplantation therapeutic agents.

When culturing pluripotent stem cells capable of serving as raw materials of cells for transplant, it is desirable to not use reagents and so forth containing raw materials derived from animals. Therefore, the development of matrices and culture media is in progress for use in culturing that satisfies such conditions (Patent Document 1 and Non-Patent Document 1).

However, studies have yet to be conducted on whether or not pluripotent stem cells cultured using such matrices or culture media have properties that are identical to pluripotent stem cells cultured according to conventional methods using reagents and so forth produced with raw materials derived from animals.

### CITATION LIST

### Patent Document

Patent Document 1: WO 2011043405

### Non-Patent Document

Non-Patent Document 1: Nakagawa, M., et al., Sci. Rep., 8; 4:3594, 2014

### SUMMARY

### Technical Problem

An object of the present invention is to provide a novel method for culturing pluripotent stem cells.

### Solution to Problem

When pluripotent stem cells were cultured on various laminins, the inventors of the present invention found that pluripotent stem cells cultured on laminin 421 or laminin 121 acquires a tendency to easily differentiate into mesodermal cells, and particularly blood cells, thereby leading to completion of the present invention.

Namely, the present invention encompasses the inventions indicated below.

[1] A method for culturing pluripotent stem cells, comprising the step of contacting pluripotent stem cells with laminin 421 or a fragment thereof, laminin 121 or a fragment thereof, or a combination thereof.

[2] The method described in [1], wherein the expression level of a gene located downstream in a Wnt/β-catenin signaling pathway and/or a gene of the IRX family is increased in the pluripotent stem cells.

[3] The method described in [2], wherein the gene located downstream in a Wnt/β-catenin signaling pathway is at least one gene selected from the group consisting of NEUROG1, PITX2, ZIC1, PAX7, HAPLN1, FOXC1, CTSF, HHEX and JUN.

[4] The method described in [2], wherein the gene of the IRX family is at least one gene selected from the group consisting of IRX4, IRX1 and IRX2.

[5] The method described in any of [1] to [4], further comprising the step of inducing differentiation of the pluripotent stem cells into mesodermal cells.

[6] The method described in [5], wherein the mesodermal cells are skeletal muscle cells, chondrocytes, renal cells, myocardial cells, vascular endothelial cells or blood cells.

[7] The method described in any of [1] to [6], wherein the fragment is an E8 fragment.

[8] The method described in any one of [1] to [7], wherein the pluripotent stem cells are human pluripotent stem cells.

[9] A kit for culturing pluripotent stem cells, comprising laminin 421 or a fragment thereof, laminin 121 or a fragment thereof, or a combination thereof.

[10] A method for producing mesodermal cells, comprising the step of inducing differentiation of the pluripotent stem cells cultured according to the method described in any of [1] to [8] into mesodermal cells.

[11] The method described in [10], wherein the mesodermal cells are skeletal muscle cells, chondrocytes, renal cells, myocardial cells, vascular endothelial cells or blood cells.

[12] The method described in [10], wherein the mesodermal cells are further induced to differentiate into megakaryocytes or megakaryocyte progenitor cells.

[13] A method for producing platelets from megakaryocytes induced to differentiate from pluripotent stem cells cultured according to the method described in any of [1] to [8].

[14] A platelet preparation containing platelets produced according to the method described in [13].

[15] A method for transplanting or transfusing platelets produced according to the method of [14] into a subject.

[16] A Wnt signaling agonist containing laminin 421 or a fragment thereof, laminin 121 or a fragment thereof, or a combination thereof.

### Advantageous Effects of Invention

According to the present invention, pluripotent stem cells that easily differentiate into mesodermal cells can be prepared by culturing in the presence of laminin 421 or laminin 121. In particular, although there is hardly any differentiation into mesodermal cells or cells end up dying without forming colonies in the case of using other laminins, culturing on laminin 421 or laminin 121 enables pluripotent stem cells to form colonies and differentiate into blood cells.

Moreover, the expression level of a gene located downstream in a Wnt/β-catenin signaling pathway or a gene of the IRX family is increased in pluripotent stem cells cultured in accordance with the present invention.

### BRIEF DESCRIPTION OF DRAWINGS

FIG. 1 shows induction results when iPS cells cultured by substituting with various laminin fragments were induced to differentiate into blood progenitor cells (CD34 and CD43-positive cells (left graph) and CD43-positive cells (right graph)).

FIG. 2 shows a growth curve of CD41-positive cells when iPS cells cultured by substituting with 421E8 or 121E8 were induced to differentiate into megakaryocyte progenitor cells followed by continuing maintenance culturing.

### DESCRIPTION OF EMBODIMENTS

(Method for Culturing Pluripotent Stem Cells)

The method for culturing pluripotent stem cells according to the present invention comprises the step of contacting pluripotent stem cells with laminin 421 or a fragment thereof, laminin 121 or a fragment thereof, or a combination thereof. A laminin fragment is used preferably.

Laminins constitute one of the important extracellular matrices that compose basement membranes and are involved in cell adhesion and so forth. Laminins are huge glycoproteins that have a large number of isoforms, each isoform forms a coiled coil structure as a result of association of each one of five types of α chains (α1, α2, α3. α4, α5), three types of β chains β1, β2, β3) and three types of γ chains (γ1, γ2, γ3) as subunits through their C terminal regions, and forms a heterotrimer molecule stabilized with disulfide bonds. Members of the laminin family are named according to the types of subunits of which they are composed. When explained using the example of laminin 511, laminin composed of an α5 chain, β1 chain and γ1 chain is referred to as laminin 511. The laminin used in the present invention is preferably laminin 421 composed of an α4 chain, β2 chain and γ1 chain and/or laminin 121 composed of an α1 chain, β2 chain and γ1 chain, or a fragment thereof such as an E8 fragment.

The laminin may be naturally-occurring or may be a modified type in which one or more, and preferably several, amino acid residues have been modified provided the biological activity thereof is maintained. There are no particular limitations on the method used to produce the laminin, and examples thereof include a method consisting of purifying from cells highly expressing laminin and a method consisting of producing laminin in the form of a recombinant protein. There are also no particular limitations on the method used to produce a laminin fragment, and examples thereof include a method consisting of digesting full-length laminin with a protease such as elastase followed by fractioning and purifying the target fragment, and a method consisting of producing in the form of a recombinant protein. Both the laminin and laminin fragment are preferably produced in the form of a recombinant protein from the viewpoints of production volume, quality uniformity and production cost.

Although there are no particular limitations on the molecular weight of the laminin fragment in the present description provided it demonstrates the effects of the present invention, the molecular weight thereof is preferably roughly equal to or greater than that of an E8 fragment. A laminin “E8 fragment” refers to a trimeric fragment consisting of a C-terminal fragment of an α chain from which globular domains 4 and 5 have been removed (hereafter referred to as “α chain E8”), a C-terminal fragment of a γ chain (referred to as “β chain E8”), and a C-terminal fragment of a γ chain (referred to as “γ chain E8”) and the molecular weight of the trimer is roughly 150 kDa to roughly 170 kDa. The α chain E8 is normally composed of about 770 amino acids and roughly 230 amino acids on the N-terminal side are involved in trimer formation. The β chain E8 is normally composed of about 220 to about 230 amino acids. The γ chain E8 is normally composed of about 240 to about 250 amino acids. The glutamic acid residue at the third position from the C-terminal of γ chain E8 is essential for the cell adhesion activity of laminin E8 (Hiroyuki Ido, Aya Nakamura, Reiko Kobayashi, Shunsuke Ito, Shaoliang Li, Sugiko Futaki and Kiyotoshi Sekiguchi, “The requirement of the glutamic acid residue at the third position from the carboxyl termini of the laminin γ chains in integrin binding by laminins”, The Journal of Biological Chemistry, 282, 11144-11154, 2007). Without intending to be bound by any theory, although the laminin fragment used in the present invention maintains an intensity of integrin binding activity that is roughly equal to or greater than that of the corresponding full-length laminin, an E8 fragment, for example, is preferable.

In the present invention, pluripotent stem cells refer to stem cells that have pluripotency enabling differentiation into all cells present in the body while also having the ability to proliferate, and include, for example, embryonic stem (ES) cells (J. A. Thomson, et al. (1998), Science 282: 1145-1147; J. A. Thomson, et al. (1995), Proc. Natl. Acad. Sci. USA, 92: 7844-7848; J. A. Thomson, et al. (1996), Biol. Reprod., 55: 254-259; J. A. Thomson and V. S. Marshall (1998), Curr. Top. Dev. Biol., 38: 133-165), embryonic stem cells derived from cloned embryos obtained by nuclear transfer (ntES cells) (T. Wakayama, et al. (2001), Science, 292: 740-743; S. Wakayama, et al. (2005), Biol. Reprod., 72: 932-936; J. Byrne, et al. (2007), Nature, 450: 497-502), germ line stem cell (“GS cells”) (M. Kanatsu-Shinohara, et al. (2003), Biol. Reprod., 69: 612-616; K. Shinohara, et al. (2004), Cell, 119: 1001-1012), embryonic germ stem cells (“EG cells”) (Y. Matsui, et al. (1992), Cell, 70: 841-847; J. L. Resnick, et al. (1992), Nature, 359: 550-551), induced pluripotent stem (iPS) cells (K. Takahashi and S. Yamanaka (2006), Cell, 126: 663-676; K. Takahashi, et al. (2007), Cell, 131: 861-872; J. Yu, et al. (2007), Science, 318: 1917-1920; Nakagawa, M., et al., Nat. Biotechnol., 26: 101-106 (2008); WO2007/069666), and pluripotent stem cells derived from cultured fibroblasts or bone marrow stem cells (Muse cells) (WO2011/007900). The pluripotent stem cells are more preferably human pluripotent stem cells. The pluripotent stem cells may be cultured in the presence of laminin 511 prior to contact with the above-mentioned laminin.

Expression levels of a gene downstream of β-catenin and/or a gene of the IRX family are increased in pluripotent stem cells cultured in the presence of a specific laminin. In particular, although expression of a gene downstream of β-catenin and/or a gene of the IRX family decreases and differentiation resistance into mesoderm is observed in pluripotent stem cells cultured on laminin 511 prior to contact with laminin 421 or laminin 121, expression of these genes increases and differentiation resistance into mesoderm is thought to be removed when cultured on laminin 421 or laminin 121.

In the case of using in the present description, a “gene located downstream in a Wnt/β-catenin signaling pathway” or a “gene downstream of β-catenin” may be a gene that interacts with β-catenin gene (CTNNB1). Such genes are known in the art and can be found by using, for example, IPA (Ingenuity Pathways Analysis)®. Without intending to be limiting and in a preferable aspect thereof, the gene downstream of β-catenin gene is at least one gene selected from the group consisting of NEUROG1, PITX2, ZIC1, PAX7, HAPLN1, FOXC1, CTSF, HHEX and JUN.

Here, the development of differentiation into mesoderm from epiblasts is known to be inhibited in mice deficient in Wnt/β-catenin signaling (Liu, P., et al., Nat. Genet. 1999; 22: 361-365, Huelsken, J., et al., J. Cell. Biol. 2000; 148: 567-578). In addition, the number of differentiating blood cells decreases when Wnt/β-catenin signaling is inhibited in blood cell differentiation using human ES cells, while conversely, the number of differentiating blood cells increases when Wnt/β-catenin signaling is activated (Woll, P. S., et al., Blood, 2008 Jan. 1; 111(1): 122-31). Without intending to be bound by any theory, these reports suggest that Wnt/β-catenin signaling is essential for differentiation into mesoderm and blood cells.

A gene of the IRX (Iroquois homeobox) family has a homeobox domain and is thought to play multiple roles during pattern formation of vertebrate embryos. In particular, IRX is known to be involved in differentiation not only to the kidneys, spleen and heart as mesodermal organs, but also in the nerves and lungs (Circ. Res., 2012; 110: 1513-1524). Although examples of the members of this gene family include Iroquois homeobox protein 1 (IRX1), IRX2, IRX3, IRX4, IRX5 and IRX6, in a preferable aspect of the present invention, the expression level of at least one gene selected from the group consisting of IRX4, IRX1 and IRX2 is increased.

In another aspect, the present invention may further comprise the step of inducing differentiation of cultured pluripotent stem cells into mesodermal cells. According to the present invention, not only can differentiation from pluripotent stem cells to mesodermal cells be induced more efficiently, induction of differentiation into blood cell groups can also be promoted. In the case of using in the present description, “mesodermal cells” or “mesoderm” refers to cells that are CD56-positive and APJ-positive. In a preferable aspect thereof, the mesodermal cells may be skeletal muscle cells, chondrocytes, renal cells, myocardial cells, vascular endothelial cells or blood cells, and are preferably megakaryocytes or progenitor cells thereof. In the present invention, blood cells refer not only to megakaryocytes or progenitor cells thereof, but also various types of blood cells including hematopoietic stem cells.

Ordinary medium used to maintain pluripotent stem cells can be used to culture and subculture pluripotent stem cells. During culturing, a protein such as vascular endothelial growth factor (VEGF), basic fibroblast growth factor (bFGF) or transforming growth factor-β (TGF-β), serum or an amino acid may be added to the medium. The culture vessel may also be coated with an extracellular matrix such as laminin 511. In addition, pluripotent stem cells can also be co-cultured with feeder cells. Any feeder cells can be used provided they are cells that contribute to growth and maintenance of the pluripotent stem cells, and C3H10T1/2 cells, for example, can be used. When using feeder cells, it is preferable to suppress cell growth by treating with mitomycin C or irradiating with radiation. However, feeder-free conditions are preferable.

The temperature during culturing of pluripotent stem cells is normally 25° C. to 39° C. and preferably 33° C. to 39° C. CO₂ concentration in the culture atmosphere is normally 4% by volume to 10% by volume and preferably 4% by volume to 6% by volume. Other culturing conditions and differentiation conditions used in the culturing method of the present invention can be suitably determined by a person with ordinary skill in the art.

In the case of preparing a net-like structure from pluripotent stem cells such as iPS cells, culturing conditions are suitably selected that are appropriate for the preparation thereof. These culturing conditions vary according to the biological species of the iPS cells or ES cells used. The presence of a net-like structure can be confirmed about 14 to 17 days after seeding on the feeder cells, for example.

(Kit)

The present invention further provides a kit for culturing pluripotent stem cells, comprising laminin 421 or a fragment thereof, laminin 121 or a fragment thereof, or a combination thereof. An example of such a kit is a culture dish coated with laminin.

The above-mentioned kit may comprise laminin 421 or a fragment thereof or laminin 121 or a fragment thereof as a Wnt signaling agonist. A Wnt signaling agonist can also be used independently separate from the kit. “Wnt signaling” refers to signaling that is activated as a result of Wnt protein acting on a cell (hereinafter to be simply referred to as “Wnt signaling”). In addition, a “Wnt signaling agonist” refers to a substance that activates Wnt signaling.

(Method for Producing Mesodermal Cells)

The method for producing mesodermal cells according to the present invention comprises the step of contacting pluripotent stem cells with laminin 421 or a fragment thereof or laminin 121 or a fragment thereof. In the case of using in the present description, “mesodermal cells” refer to cells that are CD56-positive and APJ-positive. Without intending to be limiting, mesodermal cells specifically refer to skeletal muscle cells, chondrocytes, renal cells, myocardial cells, vascular endothelial cells and blood cells (such as erythrocytes, lymphocytes or megakaryocytes). In addition, the mesodermal cells induced by the present invention are cells that have a high ability to differentiate into blood cells among cells which are CD56-positive and APJ-positive. The medium used to produce mesodermal cells may contain a component such as activin A that is required for induction of differentiation into mesodermal cells, for example. Culturing conditions preferably consist of serum-free and/or feeder-free conditions. The duration of contact is preferably 3 days or longer, for example 3 days to 5 days, and particularly 3 days to 4 days.

The mesodermal cells for which differentiation has been induced are CD56-positive and APJ-positive. CD56 and APJ have each been reported to be independent markers of mesoderm (Evseenko, D., et al., P. Natl. Acad. Sci. USA 107, 13742-β747 (2010); Vodyanik, M. A., et al., Cell. Stem Cell 7, 718-729 (2010); Yu, Q. C., et al., Blood 119, 6243-6254 (2012)). CD56 is an adhesive factor that is also known as NCAM, while APJ is a functional molecule that has been reported to be receptor for Apelin molecules and the like (APLNR).

Cells being CD56-positive and APJ-positive may further be contacted with vascular endothelial growth factor (VEGF), basic fibroblast growth factor (bFGF) and transforming growth factor beta (TGFβ) inhibitors. As a result, efficiency of differentiation from mesoderm to blood vessel progenitor cells is improved. For example, in comparison with cells being CD56-negative and APJ-negative, cells that are CD56-positive and APJ-positive are able to produce blood cells highly efficiently. An example of a TGFβ inhibitor is SB431542. Other conditions for inducing differentiation into mesodermal cells can be suitably determined by a person with ordinary skill in the art depending on the type of cell that is ultimately induced to differentiate.

In another aspect, mesodermal cells that have been induced to differentiate are further induced to differentiate megakaryocytes or megakaryocyte progenitor cells in order to produce platelets. In the present invention, “megakaryocytes” include not only multinucleated cells, but also, for example, cells characterized as CD41a-positive/CD42a-positive/CD42b-positive. In addition, megakaryocytes may also be characterized as cells that express GATA1, FOG1, NF-E2 and β1-tubulin. Multinucleated megakaryocytes refer to a cell or group of cells in which the number of nuclei has increased relatively in comparison with hematopoietic progenitor cells. For example, in the case the number of nuclei of hematopoietic progenitor cells to which the method of the present invention has been applied is 2N, then cells in which the number of nuclei is 4N or more become multinucleated megakaryocytes. In addition, in the present invention, megakaryocytes may be immortalized as a megakaryocyte cell line or may be a group of cloned cells.

In the present invention, “megakaryocyte progenitor cells” refer to cells that become megakaryocytes as a result of maturation and are not multinucleated, and include cells characterized as CD41a-positive/CD42a-positive/CD42b-weakly positive. The megakaryocyte progenitor cells of the present invention are preferably cells that can be grown by expansion culturing, and for example, are cells that can be expansion-cultured under suitable conditions for at least 60 days. In the present invention, megakaryocyte progenitor cells may or may not be cloned, and although there are no particular limitations thereon, those that have been cloned are also referred to as a megakaryocyte progenitor cell line.

In the present invention, the contact step may be carried out in the presence of cytokine when producing megakaryocyte progenitor cells. Cytokines may be contained in the culture medium. Cytokines refer to proteins that promote blood cell differentiation and examples thereof include vascular endothelial growth factor (VEGF), thrombopoietin (TPO), stem cell factor (SCF), interleukin (IL)-1, -3, -4, -6, -7 and -11, granulocyte-macrophage colony stimulating factor (GM-CSF) and erythropoietin (EPO). Preferable cytokines used in the present invention are TPO and SCF. In the case of containing TPO and SCF in the culture medium, the concentration thereof in the culture media is 10 ng/mL to 200 ng/mL and preferably about 50 ng/mL to 100 ng/mL in the case of TPO, and 10 ng/mL to 200 ng/mL and preferably about 50 ng/mL in the case of SCF.

Although there are no particular limitations thereon, the culture media used in the present invention can be prepared by using a medium used to culture animal cells as a basal medium. The definition of a basal medium includes, for example, Iscove's Modified Dulbecco's Medium (IMDM), Medium 199, Eagle's Minimum Essential Medium (EMEM), αMEM medium, Dulbecco's Modified Eagle's Medium (DMEM), Ham's F12 medium, RPMI 1640 medium, Fischer's medium, Neurobasal Medium (Life Technologies) and mixed media thereof. The medium may contain serum or a serum-free medium may be used. The basal medium can contain one or more substances such as albumin, insulin, transferrin, selenium, fatty acid, trace element, 2-mercaptoethanol, thiolglycerol, lipid, amino acid, L-glutamine, non-essential amino acid, vitamin, growth factor, low molecular weight compound, antibiotic, antioxidant, pyruvic acid, buffer, inorganic salt or cytokine as necessary.

A preferable basal medium in the present invention is IMDM medium containing serum, insulin, transferrin, serine, thiolglycerol and ascorbic acid.

In the step of producing megakaryocytes from hematopoietic progenitor cells of the present invention, an example of a method thereof consists of culturing the hematopoietic progenitor cells on feeder cells (such as cells obtained from the aorta-gonad-mesonephros (AGM) region of a mammalian fetus (Patent Publication JP-A-2001-37471), mouse embryonic fibroblasts (MEF), OP9 cells (available from ATCC) or C3H10T1/2 cells (available from JCRB Cell Bank)) or on an extracellular matrix.

In the present invention, an extracellular matrix refers to a supramolecular structure present outside a cell that may be naturally-occurring or artificial (recombinant). Examples thereof include substances in the manner of collagen, proteoglycan, fibronectin, hyaluronic acid, tenascin, entactin, elastin, fibrillin and laminin, or fragments thereof. These extracellular matrices may be used in combination and, for example, may be prepared from cells such as in the case of BD Matrigel®.

In the present invention, preferable culturing conditions for producing megakaryocyte progenitor cells consist of a method in which feeder cells in the manner of C3H10T1/2 cells are co-cultured with hematopoietic progenitor cells.

In the present invention, hematopoietic progenitor cells (HPC) refer to cells capable of differentiating into blood cells such as lymphocytes, eosinophils, neutrophils, basophils, erythrocytes or megakaryocytes. In the present invention, there is no distinction made between hematopoietic progenitor cells and hematopoietic stem cells, and indicate the same cells unless specifically specified otherwise. Hematopoietic stem cells/progenitor cells can be recognized by being positive for surface antigens CD34 and/or CD43. In the present invention, hematopoietic stem cells can also be applied to hematopoietic progenitor cells that have been induced to differentiate from pluripotent stem cells or hematopoietic stem cells and progenitor cells derived from placental blood, bone marrow blood or peripheral blood and the like. For example, in the case of using pluripotent stem cells, hematopoietic progenitor cells can be prepared from a net-like structure (also referred to as an ES-sac or iPS-sac) obtained by culturing pluripotent stem cells on C3H10T1/2 in the presence of VEGF in accordance with the method described in Takayama, N., et al., J. Exp. Med., 2817-2830 (2010). Here, a “net-like structure” refers to a steric sac-like (with a space in it) structure is derived from pluripotent stem cells, which structure is formed by an endothelial cell population and the like and contains therein hematopoietic progenitor cells. In addition, other examples of methods used to produce hematopoietic progenitor cells from pluripotent stem cells include a method employing the formation of an embryoid body and the addition of cytokine (Chadwick, et al., Blood 2003, 102: 906-15; Vijayaragavan, et al., Cell. Stem Cell 2009, 4: 248-62; Saeki, et al., Stem Cells 2009, 27: 59-67), and co-culturing with stromal cells derived from a different species (Niwa, A., et al., J. Cell. Physiol., 2009 November; 221(2): 367-77). In the present invention, preferable hematopoietic progenitor cells are hematopoietic progenitor cells derived from pluripotent stem cells.

In one aspect thereof, the method for producing megakaryocyte progenitor cells according to the present invention may comprise a step of forcibly expressing a cancer gene (such as an MYC family gene and preferably c-MYC) in hematopoietic progenitor cells, a gene that suppresses expression of p16 gene or p19 gene (such as BMI1 or Id1), and/or an apoptosis suppressor gene (such as BCL2 gene, BCL-XL gene, Survivin or MCL1), and a step of culturing the cells (Patent Publication JP-A-2015-216853).

In the present invention, although there are no particular limitations on the temperature conditions during culturing, promotion of differentiation into megakaryocyte progenitor cells is confirmed by culturing hematopoietic progenitor cells at a temperature of 37° C. or higher. Here, since a temperature of 37° C. or higher refers to a suitable temperature that does not impart damage to cells, an example thereof is a temperature of about 37° C. to about 42° C. and preferably about 37° C. to 39° C. In addition, the culturing period at a temperature of 37° C. or higher can be suitably determined by a person with ordinary skill in the art by, for example, monitoring the number of megakaryocyte progenitor cells. Although there are no particular limitations on the number of days provided the desired megakaryocyte progenitor cells are obtained, the number of days is, for example, 6 days or more, 12 days or more, 18 days or more, 24 days or more, 30 days or more, 42 days or more, 48 days or more, 54 days or more or 60 days or more, and is preferably 60 days or more. A prolonged culturing period does not present a problem in the production of megakaryocyte progenitor cells. In addition, the cells may be suitably subcultured during the culturing period.

(Platelet Production Method)

The present invention provides a method for further producing megakaryocytes and/or platelets from megakaryocyte progenitor cells obtained according to the previously described method. In the case of forcibly expressing a cancer gene, a gene suppressing expression of p16 gene or p19 gene and/or an apoptosis suppressor gene, discontinuing this forced expression and culturing the cells can produce megakaryocytes and/or platelets. In the case of forcible expression using a drug-responsive vector, for example, discontinuation of forced expression may be achieved by not contacting the cells with the corresponding drug. In addition, in the case of using a vector containing the above-mentioned LoxP, forced expression may be discontinued by introducing Cre recombinase into the cells. Moreover, in the case of using a transient expression vector and RNA or protein transduction, forced expression may be discontinued by discontinuing contact with the vector and so forth. Discontinuation of forced expression can be carried out using the same media described above when discontinuing forced expression.

Although there are no particular limitations thereon, temperature conditions when culturing after discontinuing forced expression consist of, for example, a temperature of about 37° C. to about 42° C. and preferably about 37° C. to about 39° C. In addition, although the culturing period at a temperature of 37° C. or higher can be suitably determined by a person with ordinary skill in the art by, for example, monitoring the number of megakaryocytes and the like, the culturing period is, for example, 2 days to 10 days and preferably 3 days to 7 days. The culturing period is preferably at least 3 days. In addition, the cells may be suitably subcultured during the culturing period.

In the present invention, megakaryocyte progenitor cells obtained according to the previously described method can be stored frozen. Megakaryocyte progenitor cells can be transferred and distributed while stored frozen.

In the present invention, in one aspect of the method for producing megakaryocytes and/or platelets, a ROCK inhibitor and/or actomyosin complex function inhibitor is added to the medium. An example of a ROCK inhibitor is Y27632. An example of an actomyosin complex function inhibitor is the myosin heavy chain II ATPase inhibitor, blebbistatin. ROCK inhibitor may be added to the medium alone, ROCK inhibitor and actomyosin complex function inhibitor may each be added to the medium at different times, or the two may be added in combination.

The ROCK inhibitor and/or actomyosin complex function inhibitor are preferably added to the medium at 0.1 μM to 30 μM, and more specifically, the inhibitor concentration may be, for example, 0.5 μM to 25 μM or 5 μM to 20 μM.

Although there are no particular limitations thereon, the origin of “cells” described in the present description is humans or non-human animals (such as mice, rats, cows, horses, pigs, sheep, monkeys, dogs, cats or birds), and human-derived cells are preferable.

A technology known among persons with ordinary skill in the art can be applied to the production method of the present invention with respect to the production of megakaryocytes provided the effects of the present invention are not impaired. For example, in one aspect of the method for producing megakaryocytes of the present invention, the medium may further contain: (a) a substance that inhibits the expression or function of a p53 gene product, (b) an actomyosin complex function inhibitor, (c) a ROCK inhibitor and (d) an HDAC inhibitor. These methods can be carried out in accordance with the method described in WO 2012/157586.

Moreover, the production volume of megakaryocytes can be increased by forcibly expressing a cancer gene such as c-MYC gene or an exogenous gene such as a polycomb gene as described in WO 2011/034073. In this aspect, the production method of the present invention may further comprise a step of culturing after discontinuing forced expression of megakaryocytes or megakaryocyte progenitor cells. As a method for discontinuing forced expression, in the case of forcible expression using a drug-responsive vector, for example, discontinuation of forced expression may be achieved by not contacting the cells with the corresponding drug. In addition, in the case of using a vector containing the above-mentioned LoxP, discontinuation of forced expression may be achieved by introducing Cre recombinase into the cells. Moreover, in the case of using a transient expression vector and RNA or protein transduction, forced expression may be discontinued by discontinuing contact with the vector and so forth. Discontinuation of forced expression can be carried out using the same media described above for the medium used in this step.

Platelets can be isolated from media using a method known among persons with ordinary skill in the art. Platelets obtained according to the present invention are highly safe platelets that do not express exogenous genes. Although there are no particular limitations thereon, megakaryocytes obtained in the present invention may also be expressed by, for example, an exogenous apoptosis suppressor gene or cancer gene. In this case, expression of the exogenous gene is suppressed in the platelet production step.

Platelets obtained in the present invention can be administered to a patient as a preparation. In administering the platelets, the platelets may be stored and formulated with, for example, human plasma, infusion agent, citric acid-containing physiological saline, solution having for the main agent thereof glucose-acetated Ringer's solution or platelet additive solution (PAS, Gulliksson, H., et al., Transfusion, 32: 435-440 (1992)). The storage period is about 14 days, preferably 10 days and more preferably 8 days immediately after formulation. Storage conditions preferably consist of storing while shaking and agitating at room temperature (20° C. to 24° C.).

(Platelet Transplant or Transfusion Method)

The platelet transplant or transfusion method according to the present invention comprises the step of transplanting or transfusing platelets produced according to the afore-mentioned method to a subject. Platelets produced in accordance with the method of the present invention can be transfused using the same method as that used to transfuse platelets prepared according to an ordinary method, and can be suitably administered to a subject by a person with ordinary skill in the art.

In the case of using in the present description, the term “subject” refers to any arbitrary vertebrate, including a mammal requiring platelet transplant and the like (such as a cow, pig, camel, llama, horse, goat, rabbit, sheep, hamster, guinea pig, cat, dog, rat, mouse, non-human primate (such as a cynomolgus monkey, Rhesus monkey or chimpanzee) or human). The subject may be a human or animal other than a human according to the embodiment.

Although the following provides a more detailed explanation of the present invention by indicating examples thereof, the present invention is not limited in any way by the examples.

### EXAMPLES

### Study of Effect of Culturing on Laminin 421 or Laminin 121

Human iPS cells (TKDN SeV2: iPS cells derived from human fetal skin fibroblasts established using Sendai virus) were maintained using laminin 511E8 (imatrix-511, Nippi) and StemFit (AJINOMOTO). Next, when human iPS cell colonies were co-cultured for 14 days with C3H10T1/2 feeder cells in the presence of VEGF (R&D Systems) at 20 ng/mL in accordance with the method described in Takayama, N., et al., J. Exp. Med., 2817-2830, 2010, net-like structures (sacs) were unable to be produced.

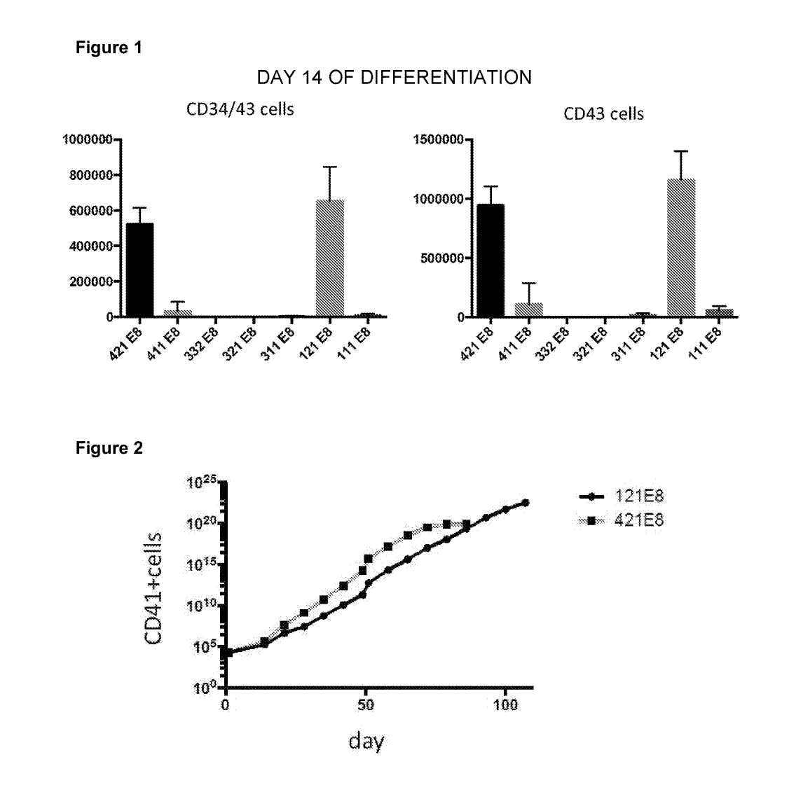

Next, after maintaining the above-mentioned iPS cells using laminin 511E8 (imatrix-511, Nippi) and StemFit (AJINOMOTO), the cells were detached using TrypLE® Select followed by transferring to culture dishes coated with each laminin fragment (111E8, 121E8, 211E8, 221E8, 311E8, 321E8, 332E8, 411E8, 421E8, 511E8 or 521E8) and culturing for 7 days. Each laminin fragment was produced using the method described in WO 2014/103534. Whereupon, iPS cell colonies were not obtained in the case of using culture dishes coated with 211E8 and 221E8. Next, in the case colonies had formed, they were co-cultured for 14 days with C3H10T1/2 feeder cells in the presence of VEGF at 20 ng/mL in the same manner as described above. As a result, net-like structures (sacs) were confirmed under conditions of coating with matrices other than 511E8 and 521E8. The resulting sacs were broken up, the suspended cells were harvested and the cells were stained using anti-CD34 antibody and anti-CD43 antibody followed by analyzing the cells using a flow cytometer. As a result, although blood progenitor cells were obtained under several conditions, numerous cells positive for CD34 and CD43 or cells positive for CD43 were obtained under conditions of coating with 421E8 and 121E8 in particular (FIG. 1)

According to the above results, it was confirmed that culturing iPS cells cultured on laminin 511E8 on 421E8 and 121E8 resulted in a change such that the ability to induce differentiation into mesodermal cells like blood cells (to be referred to as “transformation”) was achieved.

Moreover, blood progenitor cells obtained by culturing on 421E8 and 121E8 were induced to differentiate into megakaryocyte progenitor cells in accordance with the method described in Nakamura, S., et al., Cell. Stem Cell, 14: 535-548, 2014. Namely, the lentivirus method was used to forcibly express c-Myc and BMI1, followed by BCL-XL on day 14. When the obtained megakaryocyte progenitor cells were maintenance-cultured, megakaryocyte progenitor cell lines were able to be obtained for which it was possible to maintenance-culture megakaryocyte progenitor cells in the case of using either 421E8 or 121E8 (FIG. 2).

### Study of Culturing Time on Laminin 421 or Laminin 121

After having maintained human iPS cells using laminin 511E8 (imatrix-511, Nippi) and Stem Fit (AJINOMOTO) in the same manner as previously described, the cells were detached using TrypLE® Select followed by transferring to culture dishes coated with each laminin fragment (111E8, 121E8, 211E8, 221E8, 311E8, 321E8, 332E8, 411E8, 421E8 or 521E8) and culturing for 7 days (P1) or 35 days (P5). Subsequently, when differentiation into blood progenitor cells was induced in the same manner as previously described, blood progenitor cells were obtained under conditions of coating with 332E8, 421E8 and 121E8 in the case of P1. Similarly, blood progenitor cells were obtained under conditions of coating with 421E8 and 121E8 in the case of P5.

According to the above findings, the number of days of culturing on laminin 421 or laminin 121 was confirmed to not have an effect on transformation of iPS cells.

### Changes in Cells by Culturing on Laminin 421 or Laminin 121

With a transformation group (good group) (421E8 and 121E8) or non-transformation group (bad group) (111E8, 311E8, 321E8, 411E8 and 521E8) as mentioned above, iPS cells were harvested after a culture period of days at P5, and subjected to a gene expression analysis with a microarray. Among candidate genes extracted from One-way ANOVA at FDR <0.05, the presence of several candidate genes were confirmed as genes with an increased expression in the good group being more than double the expression in the bad group. Out of the several candidate genes, genes downstream of β-catenin and genes of the IRX family were excerpted and shown in Table 1.

(Gene Clusters for which Expression Increases with Transformation Group)

TABLE 1

| Gene name | Accession No. |
|---|---|
| NEUROG1 | NM_006161.2 |
| PITX2 | NM_000325.5 |
| | NM_001204397.1 |
| | NM_001204398.1 |
| | NM_001204399.1 |
| | NM_153426.2 |
| | NM_153427.2 |
| ZIC1 | NM_003412.3 |
| PAX7 | NM_001135254.1 |
| | NM_002584.2 |
| | NM_013945.2 |
| HAPLN1 | NM_001884.3 |
| FOXC1 | NM_001453.2 |
| CTSF | NM_003793.3 |
| HHEX | NM_002729.4 |
| JUN | NM_002228.3 |

TABLE 2

| | |
|---|---|
| IRX4 | NM_001278632.1 |
| | NM_001278633.1 |
| | NM_001278634.1 |
| | NM_001278635.1 |
| | NM_016358.2 |
| IRX1 | NM_024337.3 |
| IRX2 | NM_001134222.1 |
| | NM_033267.4 |

According to the above results, in the case of having re-cultured human iPS cells on 421E8 and 121E8 after culturing on laminin 511E8, expression of genes downstream of β-catenin and genes of the IRX family was confirmed to increase. Human iPS cells were suggested to reacquire the ability to induce differentiation into blood cells as a result of this change in gene expression. In addition, expression of genes downstream of β-catenin increases as a result of re-culturing on 421E8 and 121E8, which suggests that the iPS cells were converted to iPS cells with a tendency to easily differentiate into mesodermal cells.

1. A method for culturing pluripotent stem cells, comprising the step of contacting pluripotent stem cells with laminin 421 or a fragment thereof, laminin 121 or a fragment thereof, or a combination thereof.

2. The method according to claim 1, wherein the expression level of a gene located downstream in a Wnt/β-catenin signaling pathway and/or a gene of the IRX family is increased in the pluripotent stem cells.

3. The method according to claim 2, wherein the gene located downstream of β-catenin is at least one gene selected from the group consisting of NEUROG1, PITX2, ZIC1, PAX7, HAPLN1, FOXC1, CTSF, HHEX, and JUN.

4. The method according to claim 2, wherein the gene of the IRX family is at least one gene selected from the group consisting of IRX4, IRX1, and IRX2.

5. The method according to claim 1, further comprising the step of inducing differentiation of the pluripotent stem cells into mesodermal cells.

6. The method according to claim 5, wherein the mesodermal cells are skeletal muscle cells, chondrocytes, renal cells, myocardial cells, vascular endothelial cells, or blood cells.

7. The method according to claim 1, wherein the fragment is an E8 fragment.

8. The method according to claim 1, wherein the pluripotent stem cells are human pluripotent stem cells.

9. A kit for culturing pluripotent stem cells, comprising laminin 421 or a fragment thereof, laminin 121 or a fragment thereof, or a combination thereof.
